(19) United States
(12) Patent Application Publication
(10) Pub. No.: US 2009/0082738 A1
(43) Pub. Date: Mar. 26, 2009

(54) Natural Anti-Inflammatory Agents for Reducing Pain

(76) Inventor: Vijay B. Vad, New York, NY (US)

Correspondence Address: WHYTE HIRSCHBOECK DUDEK S C;INTELLECTUAL PROPERTY DEPARTMENT, 555 EAST WELLS STREET, SUITE 1900, MILWAUKEE, WI, 53202, US

(21) Appl. No.: 11/859,882

(22) Filed: Sep. 24, 2007

(51) Int. Cl. A61K 31/7008; A61F 7/08; A61K 36/87

(52) U.S. Cl. 604/291; 424/766; 514/62; 607/108

(57) ABSTRACT

An oral supplement or topical supplement containing glucosamine sulfate and oleocanthal provides relief of pain and reduces inflammation in joints. The topical supplement is used in conjunction with a heat patch.

### FIELD OF THE INVENTION

The present invention generally relates to treating pain and inflammation. More specifically, the invention relates to compounds, devices and methods for reducing inflammation of joints using natural ingredients.

### BACKGROUND OF THE INVENTION

Many conditions, such as injuries, arthritis and the like, can cause pain and inflammation in the joints of a person. A number of analgesic and/or anti-inflammation medicines are currently available for treating these symptoms. Unfortunately, these medicines can have undesirable side effects. The nature and degree of the side effects, as well as the effectiveness of the medicines, will vary on an individual case by case basis.

Anecdotally, patients frequently report fewer side effects when using natural supplements compared to standard pharmaceutical medicines. Furthermore, many people simply prefer natural ingredients and methods over medicines.

Some natural ingredients have been identified that provide relief from joint pain and inflammation. For example, clinical data on 1500 mg of glucosamine has shown that it is superior to the prescription medication Celebrex in moderate or severe knee arthritis population (Clegg et al. New England Journal of Medicine 2006). Likewise, Oleocanthal has been found to have similar anti-inflammatory properties as ibuprofen in a study published by Breslin et al. in Nature in 2005. Additionally, turmeric extract with curcumin has been shown to reduce joint inflammation in experimental rats in a study done by Funk et al. published in 2006 performed at the University of Arizona. Grape seed oil extract found in the bioflavonoid flavocoxid has been found to be superior to naproxen in knee arthritis in a trial by Schectman et al, performed in 2007.

Although these ingredients show some effectiveness, it would be advantageous to provide a method and composition that provides greater relief.

### BRIEF SUMMARY OF THE INVENTION

The present inventor has recognized that there is no current formula on the market with the combination of glucosamine sulfate, oleocanthal, grape seed oil extract, and turmeric extract containing curcumin which when used as an oral supplement or topical supplement combined with a heat patch, can create a synergistic anti-inflammatory effect on joints.

In at least some embodiments, the present invention relates to a health supplement composition comprising 1000-2000 mg of glucosamine sulfate and 50-1000 mg of oleocanthal, wherein the weights are on a per dosage basis.

In another embodiment, the invention is a device for reducing joint pain and/or inflammation, the device comprising a heat patch that comprises a composition comprising 1000-2000 mg of glucosamine sulfate and 50-1000 mg of oleocanthal, wherein the patch is adapted to transfer heat and at least some of the composition to a skin surface covering the painful or inflamed joint.

In another embodiment, the invention is a method of reducing joint pain and/or inflammation, the method comprising: administering at least one dose per day of an oral supplement comprising 1000-2000 mg of glucosamine sulfate and 50-1000 mg of oleocanthal, wherein the weights are on a per dosage basis.

In yet another embodiment, the invention is a method of reducing joint pain and/or inflammation, the method comprising: applying at least one heat patch per day to skin over the inflamed or painful joint wherein the heat patch comprises a topical supplement comprising 1000-2000 mg of glucosamine sulfate and 50-1000 mg of oleocanthal.

### DETAILED DESCRIPTION OF THE PREFERRED EMBODIMENT

The present invention was created to advance the field of joint health supplements with natural anti-inflammatory ingredients with clinical data combined to create a synergistic anti-inflammatory effect. The work was performed to have more options and solutions for managing degenerative joint conditions. The invention comprises a composition comprising glucosamine sulfate and oleocanthol, along with other optional natural ingredients. These ingredients have significant synergistic effects without the side effects seen with over-the-counter or prescription non-steroidal anti-inflammatories or cox-2 inhibitors.

Glucosamine sulfate are substances found naturally in the body. Glucosamine is a form of amino sugar that is believed to play a role in cartilage formation and repair. Glucosamine has been used extensively and studied and published extensively for pain control in arthritis. Glucosamine is thought to limit cartilage breakdown and cartilage breakdown products which can cause pain and inflammation. In some known therapies, glucosamine is combined commonly with chondroitin sulfate or methylsulfonylmethane (MSM) which enhances its ability to limit cartilage breakdown. However, even in combination, there may still not be enough pain relief from glucosamine sulfate for people wanting to be active.

Oleocanthal is a natural organic compound isolated from extra virgin olive oil, Oleocanthal is an tyrosol ester and its chemical structure [CAS No. 289030-99-5] is related to oleuropein that is also found in olive oil. Studies show that oleocanthal has anti-inflammatory and antioxidant properties and is a non-selective inhibitor of cyclooxygenase (COX). Again, however, oleocanthal may not provide enough pain and inflammation relief for active people.

In one preferred embodiment, the formula comprises of 1500 mg of glucosamine sulfate (range 1000-2000 mg) and 700 mg of oleocanthal (range 50-1000 mg) per dose. Typically, one to two doses are administered per day to provide sufficient relief to allow active people to maintain their activity levels without occurring high levels of pain or inflammation.

In other embodiments, the formula may further comprise 500 mg of grape seed oil extract (range 100-12000 mg) and/or 500 mg of turmeric extract containing curcumin (range 100-1200 mg), weights are given on a per dose basis. Grape seed oil (also called grapeseed oil or grape oil) is a vegetable oil pressed from the seeds of various varieties of Vitis vinifera grapes and is reputed to contain antioxidants such as beta-carotene, Vitamin C, Vitamin E, and omega-3 fatty acids.

The present invention can be used as an oral supplement or as a topical supplement used in conjunction with a heat patch. For use as a topical supplement, the formulation may integrated into the patch, by any convenient means such as infusion, coating the patch with a cream of liquid, or mixing with other internal ingredients of the heat patch. When the topical supplement is integrated into the material of the patch, as opposed to being a coating on the surface of the patch, at least one surface of the patch should be permeable to allow the supplement to contact the skin.

Self-heating heat patches for heating human skin are well-known. A typical heat patch generates heat via an exothermic reaction that takes place within the heat patch. In at least one embodiment, the exothermic reaction is an oxidation reaction that occurs when a mixture within the heat patch is exposed to air (usually by opening an air-tight enclosure that protects the patch during storage). The mixture may comprise loose granules of iron powder, activated carbon, water, salt, etc. and optionally a material such as wood powder for making the medium more porous.

The heat patch may be self-adhesive or may be designed to be held in place by straps or bandages.

The invention has been shown in pilot trials to have statistically significant clinical efficacy without any side effects seen with prescription anti-inflammatory medications. The inventive formulation has been tried in pilot clinical trials in recreational golfers as an oral supplement and in another group of recreational golfers as a topical applied together with a medicated heat patch for the lower back.

Experimental Trial for Oral Supplement in Knee Arthritis

A study was conducted for the present invention for oral supplements taken as two capsules twice daily with a total daily dosage of 1500 mg of glucosamine, 700 mg of oleocanthal, 500 mg of grape seed oil extract and 500 mg of turmeric extract with curmin in 17 golfers with knee arthritis. Prior to treatment with the supplements, the visual numerical scale score (0=no pain, 10=severe pain) was 7.6 (range 4-10) in the 17 golfers with knee osteoarthritis. At three months follow-up, the average visual numerical scale score was 3.8 (range 0-7). This difference was statistically significant (p<0.05). No side effects were seen due to the use of supplements.

Experimental Trial for Topical Applied with a Heat Patch

A study was conducted using a the topical cream containing 1500 mg of glucosamine, 700 mg of oleocanthal, 500 mg of grape seed oil extract and 500 mg of turmeric extract with curcumin in 12 golfers with known facet arthritis of the lumbar spine. After a round of golf without a cart, the average visual numerical pain score was 7.2 (range 5-10). The following week, the golfers each put on a 6-hour disposable adhesive heat patch containing the above topical cream on the low back. The post one round of golf without using a cart visual numerical pain score was 4.1 (range 0-8). The change in the score from with and without the medicated heat patch was statistically significant (p<0.05). No side effects were seen with the use of the medicated heat patches.

It is specifically intended that the present invention not be limited to the embodiments and illustrations contained herein, but include modified forms of those embodiments including portions of the embodiments and combinations of elements of different embodiments as come within the scope of the following claims.

I claim:

1. A health supplement composition comprising 1000-2000 mg of glucosamine sulfate and 50-1000 mg of oleocanthal, wherein the weights are on a per dosage basis.

2. The supplement of claim 1 further comprising 100-1200 mg of grape seed oil extract, wherein the weight is on a per dosage basis.

3. The supplement of claim 1 further comprising 100-1200 mg of turmeric extract containing curcumin, wherein the weight is on a per dosage basis.

4. The supplement of claim 1 further comprising 100-1200 mg of grape seed oil extract and 100-1200 mg of turmeric extract containing curcumin, wherein the weights are on a per dosage basis.

5. The supplement of claim 1, wherein the composition is in a form adapted for oral administration of the supplement.

6. The supplement of claim 5, wherein the form is a pill, tablet, caplet or capsule.

7. A device for reducing joint pain and/or inflammation, the device comprising a heat patch that comprises a composition comprising 1000-2000 mg of glucosamine sulfate and 50-1000 mg of oleocanthal, wherein the patch is adapted to transfer heat and at least some of the composition to a skin surface covering the painful or inflamed joint.

8. The device of claim 7 wherein the patch is self-adhesive to the skin surface.

9. The device of claim 7, wherein an at least semi-permeable surface of the device contacts the skin surface.

10. The device of claim 7, wherein the composition further comprises at least one of 100-1200 mg of grape seed oil extract or 100-1200 mg of turmeric extract containing curcumin.

11. The device of claim 7, wherein the heat is generated by an exothermic chemical reaction in the patch.

12. A method of reducing joint pain and/or inflammation, the method comprising:
administering at least one dose per day of an oral supplement comprising 1000-2000 mg of glucosamine sulfate and 50-1000 mg of oleocanthal, wherein the weights are on a per dosage basis.

13. The method of claim 12, wherein at least two doses are administered per day.

14. The method of claim 12, wherein the supplement further comprises at least one of 100-1200 mg of grape seed oil extract or 100-1200 mg of turmeric extract containing curcumin, wherein the weights are on a per dosage basis.

15. A method of reducing joint pain and/or inflammation, the method comprising:
applying at least one heat patch per day to skin over the inflamed or painful joint, wherein the heat patch comprises a topical supplement comprising 1000-2000 mg of glucosamine sulfate and 50-1000 mg of oleocanthal.

16. The method of claim 15, wherein the heat patch is maintained in contact with the skin for up to 6 hours.

17. The method of claim 15, wherein the heat patch is applied to the skin covering a lower back region.

18. The method of claim 15, wherein the topical supplement further comprises at least one of 100-1200 mg of grape seed oil extract or 100-1200 mg of turmeric extract containing curcumin.
